(19) United States
(12) Patent Application Publication
(10) Pub. No.: US 2012/0116436 A1
(43) Pub. Date: May 10, 2012

(54) SAFETY LANCET

(75) Inventor: Christopher Wiegel, Sunnyvale, CA (US)

(73) Assignee: ROCHE DIAGNOSTICS OPERATIONS, INC., Indianapolis, IN (US)

(21) Appl. No.: 12/939,212

(22) Filed: Nov. 4, 2010

(51) Int. Cl. A61B 17/34; B23P 11/00

(52) U.S. Cl. 606/181; 29/428

(57) ABSTRACT

A lancet, a lancing device, a method of loading a lancet into a lancing device, and methods of manufacturing and utilization thereof are disclosed. The lancet may have a needle, a body attached to the base of the needle, arm members attached to the body, and a tip cover attached to the arm members and slidably attached to the needle, wherein at least one of the tip cover and the body is selectively moveable between an operative configuration and a protective configuration. The lancing device may have a lancet holder having surfaces which retain the lancet. The method of loading a lancet may involve providing the lancet in a protective configuration and inserting the lancet into a lancet holder of the lancing device, such that the tip cover or the body is selectively moveable between an operative configuration and a protective configuration.

### TECHNICAL FIELD

The present disclosures relate generally to lancets, and particularly to lancets retained with lancing devices, and to methods of loading, manufacturing, and utilization thereof.

### BACKGROUND

The use of lancing devices in the determination of biological analyte concentration is of widespread importance, particularly to persons afflicted with type one diabetes. In regulating their disease, such diabetic patients may be required to perform an average of five to ten blood glucose tests per day via a process of self-monitoring to determine their blood glucose levels. As the number of patients suffering from diabetes increases, the practice of self-monitoring blood glucose levels has become routine. The process of self-monitoring typically involves diabetic patients obtaining a test strip, obtaining a sample of blood with a lancing device and applying the sample thereto, inserting the test strip into a blood glucose meter, and obtaining results by measuring the concentration of analytes.

During the process of self-monitoring, the risk of diabetic patients accidentally puncturing their skin with a lancing device is significant. This risk arises from both the limited physical dexterity of many diabetic patients and also from the design of lancing devices. While some attempts have been made to design lancing devices which provide protection to diabetic patients from accidental puncturing, these designs possess a variety of disadvantages. Generally, the designs require a diabetic patient to remove a safety cap from a lancet when loading the lancet into a lancing device by twisting the safety cap off of the lancet, thereby exposing the diabetic patient to the sharpened tip of a needle. Moreover, when replacing a used lancet in a lancing device, these designs further require a diabetic patient to remove the safety cap from a new lancet and place it on the used lancet, thereby exposing the diabetic patient to the sharpened tips of both the new and used needles.

### SUMMARY

In one embodiment, a lancet comprising a needle having a sharpened tip and a base remote from the sharpened tip, a body attached to the base of the needle, a pair of arm members attached to the body, and a tip cover attached to the pair of arm members and slidably attached to the needle is disclosed. In this embodiment, the tip cover forms an aperture which accommodates the sharpened tip of the needle, and wherein at least one of the tip cover and the body is selectively moveable between an operative configuration and a protective configuration. In the operative configuration, the at least one of the tip cover and the body is displaced such that the sharpened tip of the needle is exposed. In the protective configuration, the sharpened tip of the needle is concealed by the tip cover.

In another embodiment, a lancing device is disclosed of which may comprise a removable lancet and a lancet holder having retaining surfaces such that the removable lancet is retained with the lancet holder. The removable lancet may comprise a needle having a sharpened tip and a base remote from the sharpened tip, a body attached to the base of the needle, a pair of arm members attached to the body, and a tip cover attached to the pair of arm members and slidably attached to the needle, wherein the tip cover forms an aperture which accommodates the sharpened tip of the needle, and wherein at least one of the tip cover and the body is selectively moveable between an operative configuration and a protective configuration. In the operative configuration, the at least one of the tip cover and the body is displaced such that the sharpened tip of the needle is exposed. In the protective configuration, the sharpened tip of the needle is concealed by the tip cover.

In yet another embodiment, a method of providing selective exposure to and concealment of a sharpened tip of a needle which comprises utilizing the lancet according to the present invention is disclosed.

In yet still another embodiment, a method of loading a lancet into a lancing device having a lancet holder is disclosed. The method may comprise providing the lancet in a protective configuration and inserting the lancet into the lancet holder of the lancing device. The lancet may comprise a needle having a sharpened tip and a base remote from the sharpened tip, a body attached to the base of the needle, a pair of arm members attached to the body, and a tip cover attached to the pair of arm members and slidably attached to the needle. At least one of the tip cover and the body is selectively moveable between an operative configuration and the protective configuration, such that when in the operative configuration the at least one of the tip cover and the body is displaced such that the sharpened tip of the needle is exposed, and when in the protective configuration the sharpened tip is concealed by the tip cover.

In another embodiment, a method of manufacturing a lancet is disclosed, of which may comprise providing a needle having a sharpened tip and a base remote from the sharpened tip, providing a body attached to the base of the needle, providing a pair of arm members attached to the body, and providing a tip cover attached to the pair of arm members and slidably attached to the needle. The tip cover may form an aperture which accommodates the sharpened tip of the needle and at least one of the tip cover and the body may be selectively moveable between an operative configuration and a protective configuration. In the operative configuration, the sharpened tip of the needle may be exposed. In the protective configuration, the sharpened tip of the needle may be concealed by the tip cover.

These and other features and advantages of these and other various embodiments according to the present invention will become more apparent in view of the drawings, detailed description, and claims provided that follow hereafter.

### BRIEF DESCRIPTION OF THE DRAWINGS

The following detailed description of the embodiments of the present invention can be best understood when read in conjunction with the following drawings, where like structure is indicated with like reference numerals, and in which:

FIG. 1 is a front perspective view of a lancet in an operative configuration according to an embodiment of the present invention;

FIG. 2 is a front perspective view of a lancet in a protective configuration according to an embodiment of the present invention;

FIG. 3 is a front perspective view of a lancet in a protective configuration according to an embodiment of the present invention;

FIG. 4 is a front perspective view of a lancet in a transitional configuration between the protective configuration and the operative configuration according to an embodiment of the present invention;

FIG. 5 is a front perspective view of a lancet in a transitional configuration between the protective configuration and the operative configuration according to an embodiment of the present invention;

FIG. 6 is a front perspective view of a lancet in the operative configuration according to an embodiment of the present invention;

FIG. 7 is a front perspective view of a lancing device with a lancet disposed therein in a first position according to an embodiment of the present invention;

FIG. 8 is a front perspective view of a lancing device with a lancet disposed therein in a second position according to an embodiment of the present invention;

FIG. 9 is an exploded view of a lancing device according to an embodiment of the present invention;

FIG. 10 is a schematic illustration of a method of loading a lancet into a lancing device according to an embodiment of the present invention; and

FIG. 11 is a schematic illustration of a method of manufacturing a lancet according to an embodiment of the present invention.

Skilled artisans appreciate that elements in the figures are illustrated for simplicity and clarity and have not necessarily been drawn to scale. For example, the dimensions of some of the elements in the figures may be exaggerated relative to other elements, as well as conventional parts removed, to help to improve understanding of the various embodiments of the present invention.

### DETAILED DESCRIPTION

### Lancet

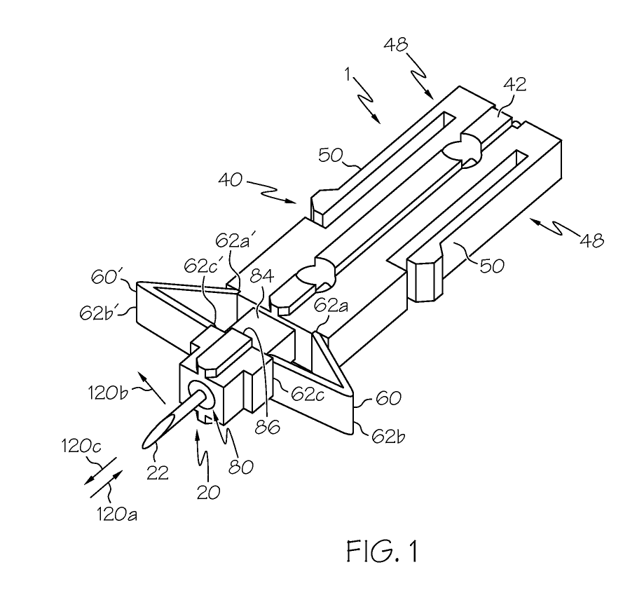

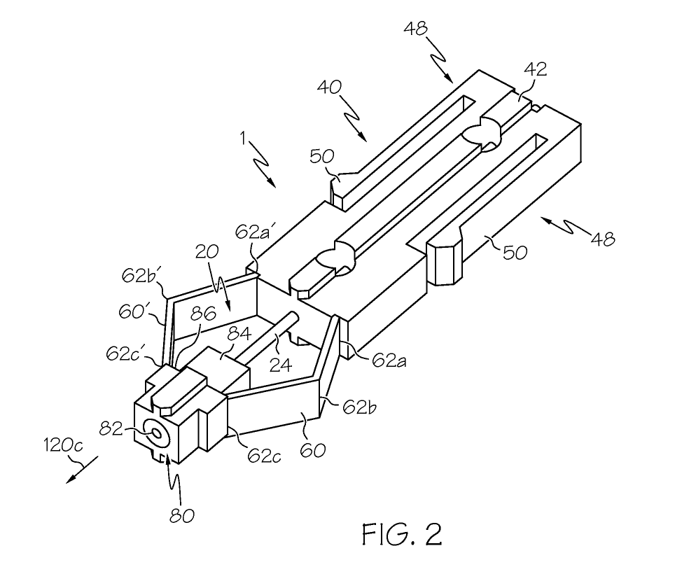

Referring to FIGS. 1 and 2, in one embodiment, a lancet 1 may comprise a needle 20, a body 40 attached to the needle 20, a pair of arm members 60, 60′ attached to the body 40, and a tip cover 80 attached to the pair of arm members 60, 60′ and slidably attached to the needle 20. In one embodiment, the needle 20 may comprises a sharpened tip 22 and a base 24 remote from the sharpened tip 22. The sharpened tip 22 of the needle 20 may comprise a shape such as for example, a substantially triangular shape, a substantially beveled edge, and/or a substantially back-cut bevel edge. However, the sharpened tip 22 of the needle 20 should not be limited to such shapes, but may comprise any shape such that the sharpened tip 22 of the needle 20 may puncture the skin and/or other tissues. In another embodiment, the base 24 of the needle 20 may comprise, but should not be limited to, a substantially flat shape or a substantially cylindrical shape.

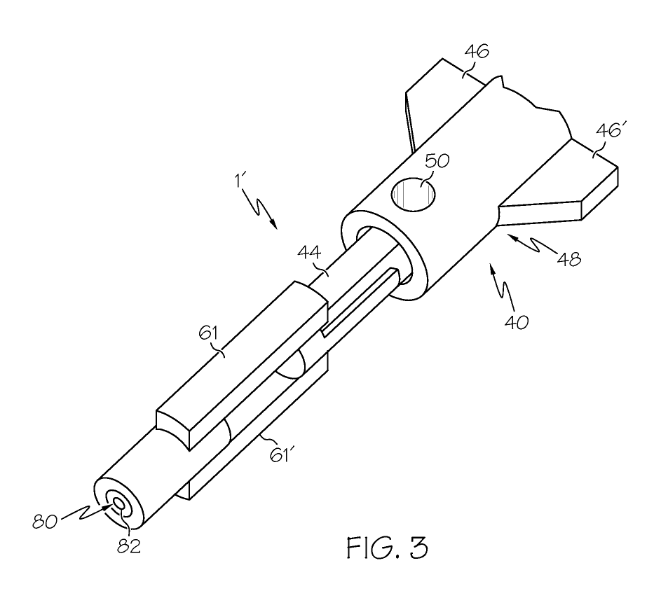

In one embodiment, the body 40 may comprise a substantially rectangular shape as depicted by FIG. 1, or a substantially cylindrical shape, such as depicted by FIG. 3. However, the shape of the body 40 should not be limited to a substantially rectangular shape or a substantially cylindrical shape, but may comprise any shape wherein the body 40 may be attached to the needle 20. In a further embodiment, the body 40 may comprise plastics, polymers, rubbers, metals, composites and/or alloys. In one particular embodiment, the body 40 may comprise polypropylene and/or polyethylene. However, the body 40 should not be limited to the materials disclosed herein, but may comprise any material wherein the body 40 is attachable to the base 24 of the needle 20.

In another embodiment, the body 40 is attached to the base 24 of the needle 20. In one particular embodiment, the body 40 is attached to the base 24 of the needle 20 via insert molding. In this embodiment, the body 40 may be molded around the base 24 of the needle 20. In another embodiment, as shown in FIGS. 1 and 2, the body 40 provides a central ridge portion 42. In one particular embodiment, the ridge portion 42 may extend through the length of the body 40. The ridge portion 42 may provide alignment and centering of the lancet 1 upon loading the lancet 1 into a lancing device (discussed below). In one particular embodiment, the lancing device (discussed below) may provide complimentary features to the ridge portion 42 of the lancet 1 for aligning and centering the lancet 1.

In one embodiment, the arm members 60, 60′ are dynamically attached to the body 40, such that they may flex outwardly in a direction substantially normal to the needle axis. In a further embodiment, as best shown in FIG. 1, the arm members 60, 60′ of the lancet 1 may comprise outwardly-flexing, substantially V-shaped arm members. However, the shape of the arm members 60, 60′ should not be limited to substantially V-shaped, but may comprise any shape wherein the arm members 60, 60′ may flex outwardly. In one embodiment, the arm members 60, 60′ may prevent the tip cover 80 from sliding off of the sharpened tip 22 of the needle 20. In another embodiment, each of the arm members 60, 60′ may comprise one or more hinges 62a, 62b, 62c, 62a′, 62b′ and 62c′, respectively, such that the arm members 60, 60′ may flex outwardly. In one embodiment, the arm members 60, 60′ may comprise only hinges 62b, 62b′ such that the tip cover 80 is biased away from the body 40. Each of the hinges 62a, 62b, 62c, 62a′, 62b′ and 62c′ may comprise a pin in hole mechanism or a living hinge mechanism or shape. However, the hinges 62a, 62b, 62c, 62a′, 62b′ and 62c′ should not be limited to those disclosed herein, but may include any hinging mechanism such that the arm members 60, 60′ attached to the body 40 may flex outwardly.

In one embodiment, the arm members 60, 60′ may flex outwardly such that the tip cover 80 translates in a direction substantially toward the body 40, exposing the sharpened tip 22 of the needle 20. In this particular embodiment, the arm members 60, 60′ may flex outwardly upon loading the lancet 1 into a lancing device (discussed below), such that the sharpened tip 22 of the needle 20 is exposed. In another embodiment, the arm members 60, 60′ may straighten from an outwardly-flexing position, such that the tip cover 80 translates in a direction substantially away from the body 40, concealing the sharpened tip 22 of the needle 20. In this particular embodiment, the arm members 60, 60′ may straighten from an outwardly-flexing position upon removing the lancet 1 from a lancing device (discussed below), such that the sharpened tip 22 of the needle 20 is concealed.

In a further embodiment, the arm members 60, 60′ may comprise plastics, polymers, rubbers, metals, composites and/or alloys. The arm members 60, 60′ may comprise a material that is different from the material of the body 40. For example, in one particular embodiment, the arm members 60, 60′ may comprise a silicone rubber overmold. In another embodiment, the arm members 60, 60′ may comprise a material that is the same as the material of the body 40. For example, in one embodiment, the arm members 60, 60′ may comprise polypropylene and/or polyethylene. However, the arm members 60, 60′ should not be limited to the materials disclosed herein, but may comprise any material wherein the arm members 60, 60′ may be dynamically or statically attached to the body 40 as described above.

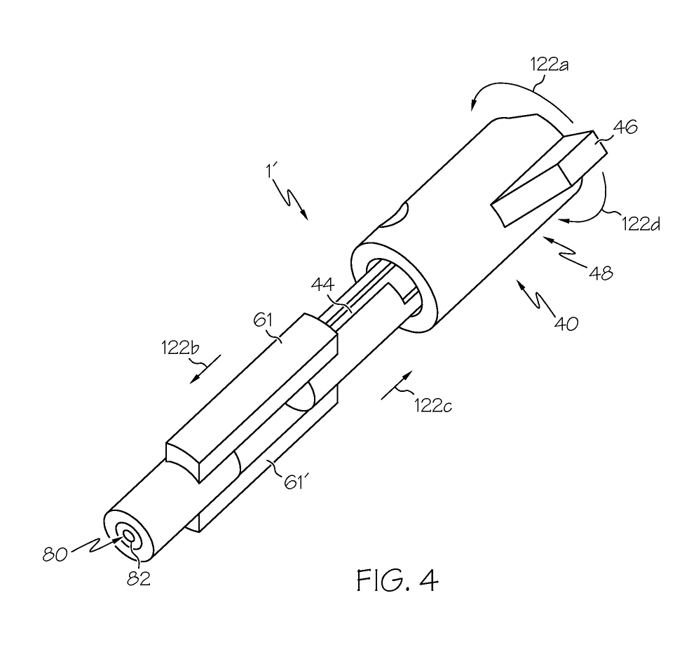

In another embodiment, as shown in FIGS. 1 and 2, the tip cover 80 may attached to the pair of arm members 60, 60′ and slidably attached to the needle 20. In one particular embodiment, the tip cover 80 may comprise a substantially rectangular shape as depicted in FIGS. 1 and 2, or a substantially cylindrical shape as depicted in FIGS. 3 and 4. However, the shape of the tip cover 80 should not be limited to those described herein, but may comprise any shape wherein the tip cover 80 is attached to the pair of arm members 60, 60′ and is slidably attached to the needle 20.

In a further embodiment, the tip cover 80 forms an aperture 82 which accommodates the sharpened tip 22 of the needle 20. In one particular embodiment, the cross-sectional shapes of the aperture 82 and the needle 20 are substantially circular. However, the cross-sectional shapes of the aperture 82 and the needle 20 should not be limited to substantially circular, but may comprise any cross-sectional shape wherein the tip cover 80 is slidably attached to the needle 20 and accommodates the sharpened tip 22 of the needle 20 through the aperture 82. In a further embodiment, the tip cover 80 is slidably attached to the needle 20 via a frictional fit.

In one embodiment, the tip cover 80 may comprise plastics, polymers, rubbers, metals, composites and/or alloys. The tip cover 80 may comprise a material that is different from the material of the body 40. For example, in one particular embodiment, the tip cover 80 may comprise polypropylene and/or polyethylene. However, the tip cover 80 should not be limited to the materials disclosed herein, but may comprise any material wherein the tip cover 80 may be attached to the pair of arm members 60, 60′, may be slidably attached to the needle 20, and may accommodate the sharpened tip 22 of the needle 20.

In another embodiment, as shown in FIGS. 1 and 2, the lancet 1 may further comprise a stopper member 84 attached to the tip cover 80 and slidably attached to the needle 20. In one particular embodiment, the stopper member 84 is substantially rectangular. However, the shape of the stopper member 84 should not be limited to substantially rectangular, but may comprise any shape wherein the stopper member 84 is slidably attached to the needle 20. In a further embodiment, the stopper member 84 is attached to or integral with the base 86 of the tip cover 80. In still a further embodiment, the stopper member 84 may comprise a channel (not shown), such that the stopper member 84 is slidably attached to the needle 20 through the channel.

In one embodiment, the stopper member 84 may comprise plastics, polymers, rubbers, metals, composites and/or alloys. Where the stopper member 84 is attached to the tip cover 80, the stopper member 84 may comprise a material that is different from the material of the tip cover 80. In one embodiment, the stopper member 84 may comprise an elastomer. In a further embodiment, the stopper member 84 may comprise silicone rubber. However, the stopper member 84 should not be limited to the materials disclosed herein, but may comprise any material wherein the stopper member 84 may limit the translation of the tip cover 80 toward the body 40, as discussed below.

In another embodiment, at least one of the tip cover 80 and the body 40 is selectively moveable between an operative configuration and a protective configuration. As the operative and protective configurations are discussed further in later sections, no further details are provided now, but instead another embodiment of the lancet is discussed hereafter with reference made to FIGS. 3 and 4. In the interest of brevity, only differences from previously discussed embodiments will be described in detail hereinafter.

As shown in FIGS. 3 and 4, one of the differences in this alternative embodiment of the lancet, generally referenced by symbol 1′, is that the body 40 forms a guide track 44 such that the body 40 is slidably engageable with a pair of followers or following arm members 61, 61′ through the guide track 44. In one particular embodiment, the guide track 44 may comprise a substantially rectangular shape. In another embodiment, the guide track 44 may comprise a substantially helical shape, such that the following arm members 61, 61′ will rotate relative to the body 40 when moving linearly along the guide track 44. However, the guide track 44 may comprise any shape such that it may complimentarily engage the pair of following arm members 61, 61′. In this embodiment, the guide track 44 extends through at least a portion of the body 40, such that the body 40 is engageable with the pair of following arm members 61, 61′.

Still referring to FIGS. 3 and 4, in a further embodiment, the body 40 of the lancet 1′ may comprise a pair of gripping members 46, 46′. The gripping members 46, 46′ are used to provide lancet users the ability to easily grasp the body 40 of the lancet 1. In one particular embodiment, the gripping members 46 may comprise a substantially wing-like shape. However, the shape of the gripping members 46 should not be limited to substantially wing-like, but may comprise any shape wherein lancet users may easily grasp the body 40. In another embodiment, the gripping members 46 are arranged on substantially opposing sides of the body 40. However, the gripping members 46 may comprise any arrangement on the body 40 such that they may be easily grasped by lancet users.

In another embodiment, as shown in FIG. 3, the following arm members 61, 61′ of the lancet 1′ are statically attached to the body 40, such that they are substantially stationary. In this particular embodiment, the following arm members 61, 61′ may comprise a pair of substantially rectangular members. In another embodiment, the following arm members 61, 61′ may comprise a substantially arcuate shape. However, the following arm members 61, 61′ may comprise any shape wherein the following arm members 61, 61′ are substantially stationary and may slidably engage the guide track 44. A discussion of the operative and protective configurations of the embodiments of the lancet 1, 1′ are now provide hereafter.

Operative Configuration

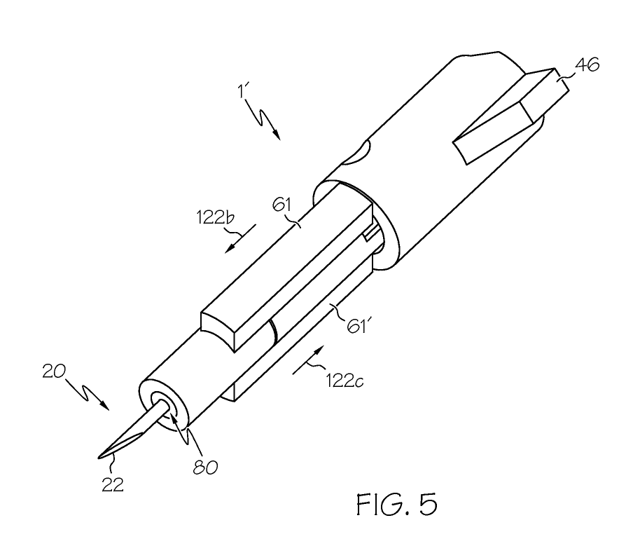

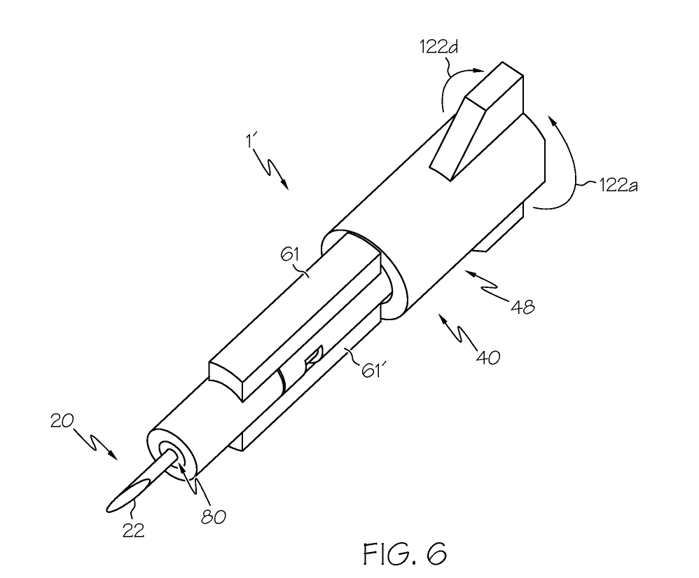

As shown in FIGS. 1, 5, and 6, in the operative configuration, the at least one of the tip cover 80 and the body 40 of the lancet 1, 1′ is displaced such that the sharpened tip 22 of the needle 20 is exposed. In one particular embodiment of the operative configuration (i.e. the translational configuration), as shown in FIG. 1, the tip cover 80 of the lancet 1 is selectively moveable between the operative configuration and the protective configuration, such that when in the operative configuration, the tip cover 80 is displaced. In this embodiment, displacement of the tip cover 80 may comprise the pair of arm members 60, 60′ flexing outwardly in a directly substantially normal to the axis of the needle 20 such that the tip cover 80 translates in a direction substantially toward the body 40 (as indicated by arrows 120a and 120b, respectively, in FIG. 1). In this embodiment, the tip cover 80 translates in a direction substantially toward the body 40 such that the stopper member 84 also translates in a direction substantially toward the body 40. In a further embodiment, the tip cover 80 translates in a direction substantially toward the body 40 until the stopper member 84 engages the body 40. By engaging the body 40, the stopper member 84 limits the translation of the tip cover 80 toward the body 40 in the operative configuration.

In an alternative embodiment of the operative configuration (i.e. the rotary configuration), as shown in FIGS. 4, 5, and 6, the body 40 of the lancet 1′ is selectively moveable between the operative configuration and the protective configuration, such that when in the operative configuration the body 40 is displaced. In this particular embodiment, displacement of the body 40 may comprise the body 40 rotating and translating relative to the pair of following arm members 61, 61′. In this embodiment, the body 40 rotates in a substantially radial direction and translates in a direction substantially toward the tip cover 80 (as indicated by arrows 122a and 122b, respectively, in FIGS. 4, 5, and 6). More particularly, the body 40 rotates such that the guide track 44 is substantially aligned with the pair of following arm members 61, 61′. In one specific embodiment, the body 40 rotates approximately 90° such that the guide track 44 is substantially aligned with the pair of following arm members 61, 61′. When the guide track 44 is substantially aligned with the pair of following arm members 61, 61′, the body 40 slidably engages the pair of following arm members 61, 61′ such that the body 40 translates in a direction substantially toward the tip cover 80.

In the operative configuration, a portion of the needle 20 is exposed such that the sharpened tip 22 of the needle 20 may puncture the skin and/or other tissues. In one particular embodiment, a portion of the needle 20 is exposed such that the sharpened tip 22 of the needle 20 may puncture the skin and/or other tissues to an appropriate depth. Moreover, in the operative configuration, a portion of the needle 20 is exposed at a substantially constant distance from the tip cover 80. In one particular embodiment, the needle 20 is exposed at a distance from the tip cover 80 ranging from about 0.5 mm to about 2.5 mm. In another embodiment, the needle 20 is exposed at a distance from the tip cover 80 ranging from about 0.5 mm to about 1.5 mm. In one specific embodiment, the needle 20 is exposed at a distance from the tip cover 80 of about 1.5 mm.

Protective Configuration

As shown in FIGS. 2, 3, and 4, in the protective configuration, the sharpened tip 22 of the needle 20 is concealed by the tip cover 80. Referring particularly to FIG. 2, in one embodiment of the protective configuration, the protective configuration may comprise selective fixation of the tip cover 80 of the lancet 1. Referring to FIGS. 3 and 4, in another embodiment of the protective configuration, the protective configuration may comprise selective fixation of the body 40 of the lancet 1′.

In yet another embodiment, the lancet 1, 1′ may comprise retaining surfaces 48. In this particular embodiment, the lancet 1, 1′ may comprise retaining surfaces 48 such that the lancet 1, 1′ is releasably retainable with a lancing device having a lancet holder, as discussed below. In this particular embodiment, the lancing holder, as discussed below, may comprise a first position and a second position. The first position provides the operative configuration of the lancet 1, 1′ and the second position provides the protective configuration of the lancet 1, 1′.

In another embodiment, a method of providing selective exposure to and concealment of the sharpened tip 22 of a needle 20 comprises utilizing a lancet 1, 1′ according any one of the various embodiments described and exemplified herein.

Lancing Device

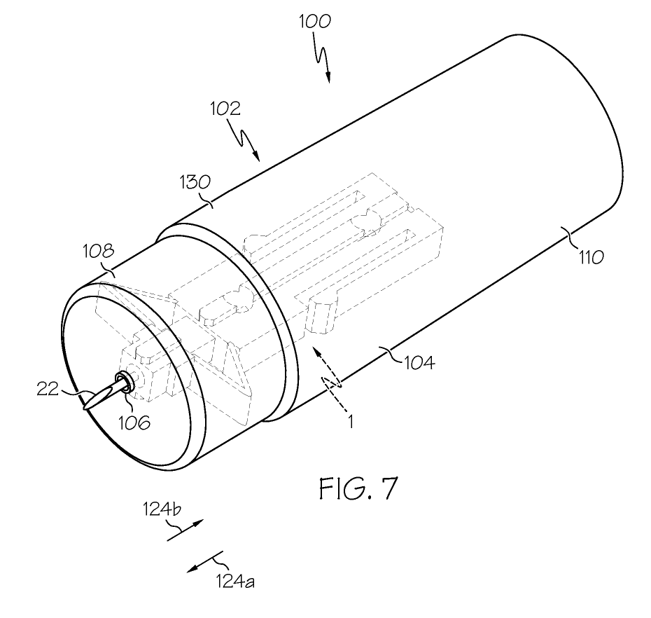

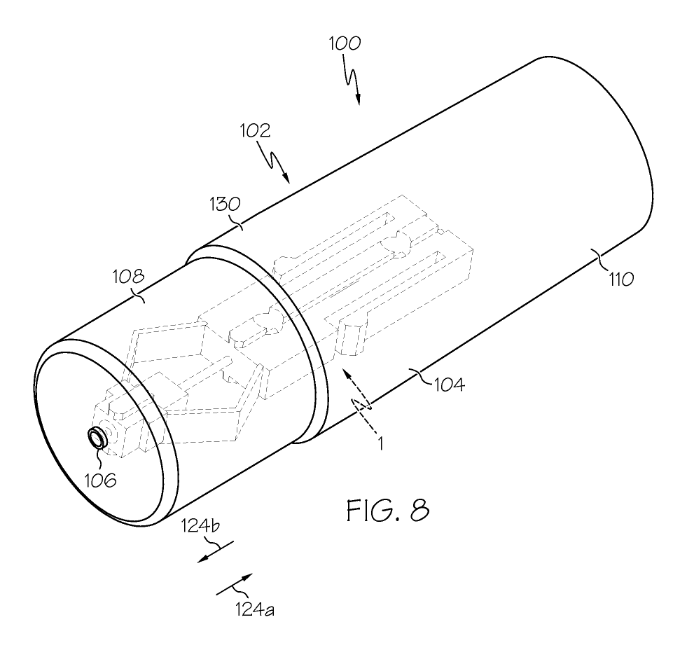

Referring now to FIGS. 7, 8, and 9, in another embodiment, a lancing device 100 is depicted comprising a lancet 1, 1′ according to any one of the various embodiments described and exemplified herein. In one embodiment, the lancing device 100 comprises a receptacle or lancet holder 102 having surfaces 104 such that the lancet 1, 1′ is releasably retained and accommodated within the lancet holder 102 (as shown by dashed lines in FIGS. 7 and 8). In another embodiment, the lancing device 100 may comprise a receptacle or lancet holder 102 having surfaces 104 such that the lancet 1, 1′ is mounted with the lancet holder 102. In one particular embodiment, the surfaces 104 of the lancet holder 102 are complimentary to the body 40, the pair of arm members 60, 60′ attached to the body 40, and the tip cover 80 attached to the pair of arm members 60, 60′ of the lancet 1, 1′, such that the lancet 1, 1′ is complimentarily retained and accommodated within the lancet holder 102. In a further embodiment, the surfaces 104 of the lancet holder 102 are complimentary to the gripping members 46 of the lancet 1, 1′, such that the lancet 1, 1′ is complimentarily retained and accommodated within the lancet holder 102.

In a further embodiment, the lancet 1, 1′ is releasably retained within the lancet holder 102 via a fastening mechanism (not shown) which secures the lancet 1, 1′ to the lancet holder 102 of the lancing device 100. The fastening mechanism may comprise a spring bias catch mechanism, a snap-fit mechanism, a frictional fit mechanism, or a latching mechanism. The lancet 1, 1′ may also be retained within the lancet holder 102 via latches and hooks, detents and ribs, and/or magnets. However, the fastening mechanism should not be limited to the mechanisms disclosed herein, but may comprise any mechanism capable of releasably retaining the lancet 1, 1′ with the lancet holder 102.

In one particular embodiment, as shown in FIG. 1, the retaining surfaces 48 of the lancet 1 may comprise interlocking members 50 which may securely mate with complimentary members (not shown) of the receiving surfaces 104 of the lancet holder 102. In this particular embodiment, the interlocking members 50 securely mate with the complimentary members of the lancet holder 102 by slidably engaging the complimentary members. In this embodiment, the lancet 1 is releasably retained with the lancet holder 102 via a frictional fit mechanism.

In another embodiment, referring to FIGS. 3 and 4, the gripping members 46 of the lancet 1′ and the receiving surfaces 104 of the lancet holder 102 provide a lock and key mechanism. In this embodiment, the gripping members 46 securely mate with the receiving surfaces 104 of the lancet holder 102, such that the lancet holder 102 may not provide the first position until the gripping members 46 properly engage the receiving surfaces 104 of the lancet holder 102. Thus, the gripping members 46 prevent the lancet holder 102 from providing the first position until the lancet 1′ is properly loaded into the lancet holder 102.

In a further embodiment, the lancet holder 102 forms an aperture 106 which accommodates the sharpened tip 22 of the needle 20. In one particular embodiment, the cross-sectional shapes of the aperture 106 and the needle 20 are substantially circular. However, the cross-sectional shapes of the aperture 106 and the needle 20 should not be limited to substantially circular, but may comprise any cross-sectional shape wherein the lancet holder 102 accommodates the sharpened tip 22 of the needle 20 through the aperture 106, providing the operative configuration.

In one embodiment, the lancet holder 102 may comprise plastics, polymers, rubbers, metals, composites and/or alloys. The lancet holder 102 may comprise a material that is different from the material of the lancet 1, 1′. For example, in one particular embodiment, the lancet holder 102 may comprise polymers. In a further embodiment, the lancet holder 102 may comprise acrylonitrile butadiene styrene, polycarbonate, and/or acetal copolymer. However, the lancet holder 102 should not be limited to the materials disclosed herein, but may comprise any material wherein the lancet holder 102 may releasably retain the lancet 1, 1′.

In a further embodiment, the lancet holder 102 may comprise a first position and a second position. In this particular embodiment, the lancet holder 102 is selectively moveable between the first position and the second position, wherein the first position provides the operative configuration of the lancet 1, 1′ and the second position provides the protective configuration of the lancet 1, 1′. When in the first position, at least one of the tip cover 80 and the body 40 is displaced such that the sharpened tip 22 of the needle 20 is exposed.

Referring specifically to FIGS. 7 and 8, in one embodiment, the lancing device 100 may comprise a lancet holder 102 and a lancet 1 in the translational configuration. In this particular embodiment wherein the lancet holder 102 selectively moves from the second position to the first position, the lancet holder 102 may comprise a cover 108 slidably attached to a body 110. In one particular embodiment, the cover 108 may be biased away from the body 110 under springs (not shown). In this embodiment, the cover 108 may translate in a direction substantially toward the body 110 (as shown by arrow 124a in FIGS. 7 and 8) when the user applies pressure to the cover 108 of the lancet holder 102 by pressing against the skin and/or other tissues. In this way, the translation of the cover 108 toward the body 110 exposes the sharpened tip 22 of the needle 20 such that the skin and/or other tissues may be punctured.

In this embodiment, the lancet holder 102 may selectively move from the first position to the second position wherein the cover 108 translates in a direction substantially away from the body 110 under spring-bias. In this embodiment, the cover 108 may translate under spring-bias following the release of pressure against the skin and/or other tissues of the user.

In another embodiment wherein the lancet holder 102 selectively moves from the second position to the first position, the lancet holder 102 may comprise a cover 108 slidably attached to a body 110 and the cover 108 may be biased away from the body 110 under springs (not shown). In this embodiment, the cover 108 may translate in a direction substantially toward the body 110 until the cover 108 engages a detent position (not shown). The cover 108 may provide a detent (not shown) which may slidably engage a recess (not shown) provided by the body 110 such that the cover 108 may translate in a direction substantially toward the body 110 until the detent engages the recess (not shown). In another embodiment, the body 110 may provide a detent (not shown) which may slidably engage a recess (not shown) provided by the cover 108 such that the cover 108 may translate in a direction substantially toward the body 110 until the detent engages the recess (not shown). In this way, the translation of the cover 108 toward the body 110 is limited, exposing the sharpened tip 22 of the needle 20 such that the skin and/or other tissues may be punctured.

In this embodiment wherein the lancet holder 102 selectively moves from the first position to the second position, the cover 108 may translate in a direction substantially away from the body 110 after the detent is released from its engagement with the recess. For example, in one embodiment, the lancet holder 102 may be provided with a barrel 130 rotatably mounted on the body 110. In one embodiment, the barrel 130 may be operably connected to the portion of the body 110 which provides the recesses (not shown) and/or the detents (not shown). In this way, the barrel 130 may unseat the detents from the recesses upon rotation such that the cover 108 springs forward in a direction substantially away from the body 110. In one embodiment, the barrel 130 may be rotated in a substantially radial direction to a variety of fixed points. In another embodiment, the barrel 130 may provide a grippable surface such that the user may easily rotate the barrel 130 in a substantially radial direction.

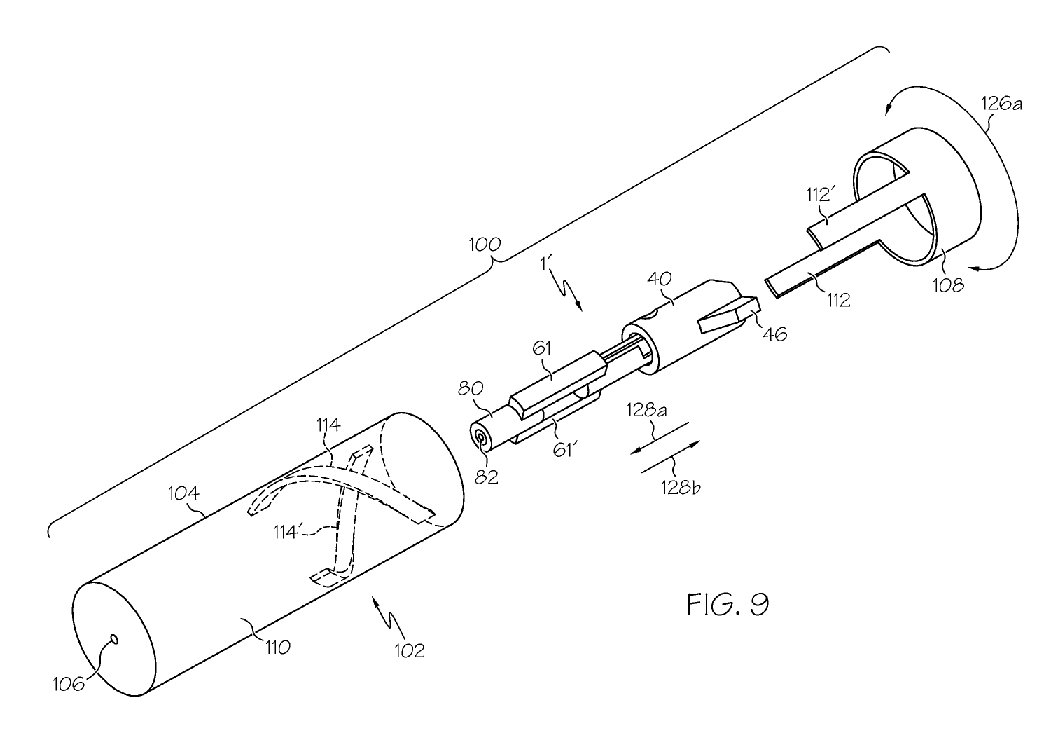

Referring specifically to FIG. 9, in another embodiment, the lancing device 100 may comprise a lancet holder 102 and a lancet 1′ in the rotary configuration. In this particular embodiment wherein the lancet holder 102 selectively moves from the second position to the first position, the lancet holder 102 may comprise a cover 108 rotatably attachable to a body 110. In one embodiment, the cover 108 may comprise prongs 112, 112′ wherein the cover 108 may engage the lancet 1′ therebetween. In another embodiment, the receiving surfaces 104 may define a pair of parallel tracks 114, 114′ on the interior of the body 110 for slidably receiving the lancet 1′. In one particular embodiment, the tracks 114, 114′ may slidably receive the gripping members 46, 46′. In another embodiment, the tracks 114, 114′ may comprise a substantially helical shape. In this embodiment, the tip cover 80 of the lancet 1′ may be releasably retained with the lancet holder 102 via a fastening mechanism (not shown). In one particular embodiment, the tip cover 80 of the lancet 1′ may be releasably retained with the lancet holder 102 via a rubber gasket substantially surrounding the tip cover 80, creating a frictional fit. In this particular embodiment, the tip cover 80 of the lancet 1′ may be selectively fixed. In another embodiment, the body 40 of the lancet 1′ may be biased away from the tip cover 80 of the lancet 1′ with springs (not shown).

In one embodiment, the lancet holder 102 selectively moves from the second position to the first position wherein the cover 108 is rotated in a substantially radial direction (i.e. forward rotation) about its attachment to the body 110 (as shown by double sided arrow 126a in FIG. 9). In one embodiment, the cover 108 may be rotated from about 0° to about 180°. In one particular embodiment, the cover 108 may be rotated from about 0° to about 90°.

In one particular embodiment, the gripping members 46, 46′ may slide along the tracks 114, 114′ in a direction substantially toward the aperture 106 of the body 110 (as shown by arrow 128a in FIG. 9) upon rotation of the cover 108. In one particular embodiment wherein the tip cover 80 of the lancet 1′ is selectively fixed, the body 40 is displaced relative to the tip cover 80 as previously described and exemplified, exposing the needle 20 such that the sharpened tip 22 of the needle 20 may puncture the skin and/or other tissues as previously discussed.

In another embodiment wherein the body 40 of the lancet 1′ is biased away from the tip cover 80 of the lancet 1′ with springs (not shown), the tip cover 80 of the lancet 1′ may be translationally fixed but may be free to rotate about its attachment to the body 40. In this way, the body 40 and the tip cover 80 may rotate relative to each other such that the pair of arm members 61, 61′ may slidably engage the guide track 44. Upon the arm members 61, 61′ engaging the guide track 44, the body 40 is displaced as previously described and exemplified, exposing the needle 20 such that the sharpened tip 22 of the needle 20 may puncture the skin and/or other tissues as previously discussed.

In another embodiment wherein the lancet holder 102 selectively moves from the first position to the second position, the cover 108 of the lancet holder 102 may be rotated in a direction substantially opposite to its forward rotation about its attachment to the body 110 (as shown by double sided arrow 126a in FIG. 9) such that the gripping members 46, 46′ slide along the tracks 114, 114′ in a direction substantially away from the aperture 106 of the body 110 (as shown by arrow 128b in FIG. 9). In one embodiment wherein the body 40 is displaced relative to the tip cover 80, rotation of the cover 108 of the lancet holder 102 causes the body 40 to translate relative to the tip cover 80 in a direction substantially away from the tip cover 80. In this way, the sharpened tip 22 of the needle 20 is concealed. In another embodiment wherein the body 40 and the tip cover 80 may rotate relative to each other, the body 40 may be biased away from the tip cover 80 such that rotation of the cover 108 causes the body 40 to translate in a direction substantially away from the tip cover 80 under spring bias. In this way, the body 40 and the tip cover 80 may rotate relative to each other and the body 40 may translate away from the tip cover 80 such that the sharpened tip 22 of the needle 20 is concealed.

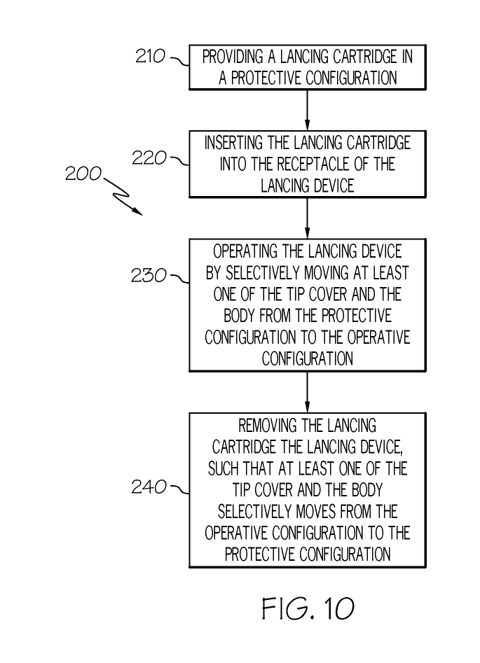

In another embodiment, as shown in FIG. 10. the present invention relates to a method of loading 200 a lancet 1, 1′ into a lancing device 100 having a lancet holder 102, the method comprising providing 210 the lancet 1, 1′ in the protective configuration and inserting 220 the lancet 1, 1′ into the lancet holder 102 of the lancing device 100. The lancet 1, 1′ may comprise a needle 20 having a sharpened tip 22 and a base 24 remote from the sharpened tip 22, a body 40 attached to the base 24 of the needle 20, a pair of arm members 60, 60′, 61, 61′ attached to the body 40, and a tip cover 80 attached to the pair of arm members 60, 60′, 61, 61′ and slidably attached to the needle 20, wherein at least one of the tip cover 80 and the body 40 is selectively moveable between the operative configuration and the protective configuration. In the operative configuration, at least one of the tip cover 80 and the body 40 is displaced relative to each other such that the sharpened tip 22 of the needle 20 is exposed. In the protective configuration, the sharpened tip 22 is concealed by the tip cover 80. In this embodiment, the lancet 1, 1′ is inserted into the lancet holder 102 of the lancing device 100 such that at least one of the tip cover 80 and the body 40 is selectively moveable from the protective configuration to the operative configuration, exposing the sharpened tip 22 of the needle 20.

In one embodiment, the lancet 1, 1′ is as previously described and exemplified. In another embodiment, the lancet 1, 1′ is inserted 220 into the lancet holder 102 of the lancing device 100. The lancet holder 102 is as previously described and exemplified. In one particular embodiment wherein the lancet 1 comprises the translational configuration, the lancet 1 is inserted 220 into the lancet holder 102 of the lancing device 100 such that the tip cover 80 is displaced as described and exemplified above. In this embodiment, the tip cover 80 is displaced when the tip cover 80 engages the receiving surfaces 104 of the lancet holder 102, causing the tip cover 80 to translate via flexing of the pair of arm members 60, 60′ in a direction substantially toward the body 40, exposing the sharpened tip 22 of the needle 20.

In another embodiment wherein the lancet 1′ comprises the rotary configuration, the body 40 is displaced such that the sharpened tip 22 of the needle 20 is exposed. In this embodiment wherein the lancet 1′ is inserted into the lancet holder 102 of the lancing device 100, the body 40 is displaced when the body 40 is rotated (i.e. forward rotation) and translated relative to the pair of arm members 61, 61′. More particularly, the body 40 is rotated in a substantially radial direction and translated in a direction substantially toward the tip cover 80, as described and exemplified above. In this particular embodiment, the body 40 is rotated and translated such that it engages the receiving surfaces 104 of the lancet holder 102, exposing the sharpened tip 22 of the needle 20.

As previously discussed, the lancet holder 102 is selectively moveable between the first position and the second position, wherein the first position provides the operative configuration of the lancet 1, 1′ and the second position provides the protective configuration of the lancet 1, 1′. The lancet holder 102 is selectively moveable between the first position and the second position as previously discussed. In another embodiment, the method may further comprises operating 230 the lancet holder 102 by selectively moving at least one of the tip cover 80 and the body 40 from the protective configuration to the operative configuration, exposing the sharpened tip 22 of the needle 20, as previously discussed.

In a further embodiment, the lancet 1, 1′ is removed 240 from the lancet holder 102 in the protective configuration, such that the sharpened tip 22 is concealed by the tip cover 80. In this particular embodiment, at least one of the tip cover 80 and the body 40 reverses its displacement from the protective configuration to the operative configuration, such that the lancet 1, 1′ is removed from the lancet holder 102 in the protective configuration. In one particular embodiment wherein the lancet 1 comprises the translational configuration, removal of the lancet 1 requires the tip cover 80 to disengage the receiving surfaces 104 of the lancet holder 102, such that the tip cover 80 translates via flexing of the pair of arm members 60, 60′ in a direction substantially away from the body 40 (as indicated by arrow 120c in FIGS. 1 and 2), concealing the sharpened tip 22 of the needle 20. In another embodiment wherein the lancet 1′ comprises the rotary configuration, removal of the lancet 1′ requires the body 40 to translate and rotate relative to the pair of arm members 61, 61′. More particularly, removal of the lancet 1′ requires the body 40 to translate in a direction substantially away from the tip cover 80 and rotate in a substantially radial direction opposite to that of its forward rotation (as indicated by arrows 122c and 122d respectively in FIGS. 4, 5, and 6). In this way, the lancet 1′ is removed from the lancet holder 102 in the protective configuration.

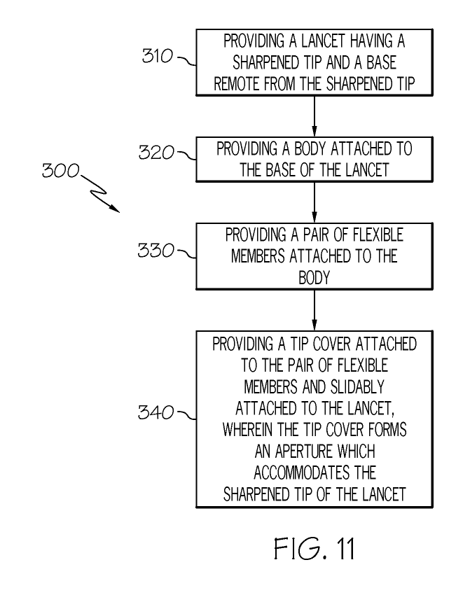

In still another embodiment, as shown in FIG. 11, the present invention relates to a method of manufacturing 300 a lancet 1, 1′ comprising providing 310 a needle 20 having a sharpened tip 22 and a base 24 remote from the sharpened tip 22, providing 320 a body 40 attached to the base 24 of the needle 20, providing 330 a pair of arm members 60, 60′, 61, 61′ attached to the body 40, and providing 340 a tip cover 80 attached to the pair of arm members 60, 60′, 61, 61′ and slidably attached to the needle 20, wherein the tip cover 80 forms an aperture 82 which accommodates the sharpened tip 22 of the needle 20, and wherein at least one of the tip cover 80 and the body 40 is selectively moveable between an operative configuration and a protective configuration. In the operative configuration, the sharpened tip 22 of the needle 20 is exposed. In the protective configuration, the sharpened tip 22 of the needle 20 is concealed by the tip cover 80.

For the purposes of describing and defining the present invention it is noted that the terms “about” and “substantially” are utilized herein to represent the inherent degree of uncertainty that may be attributed to any quantitative comparison, value, measurement, or other representation. The terms “about” and “substantially” are also utilized herein to represent the degree by which a quantitative representation may vary from a stated reference without resulting in a change in the basic function of the subject matter at issue.

The above description and drawings are only to be considered illustrative of exemplary embodiments, which achieve the features and advantages of the present invention. Modification and substitutions the features and steps described can be made without departing from the intent and scope of the present invention. Accordingly, the invention is not to be considered as being limited by the foregoing description and drawings, but is only limited by the scope of the appended claims.

What is claimed is:

1. A lancet comprising:
a needle having a sharpened tip and a base remote from the sharpened tip;
a body attached to the base of the needle;
a pair of arm members attached to the body; and
a tip cover attached to the pair of arm members and slidably attached to the needle, wherein the tip cover forms an aperture which accommodates the sharpened tip of the needle, and wherein at least one of the tip cover and the body is selectively moveable between an operative configuration and a protective configuration such that:
when in the operative configuration the at least one of the tip cover and the body is displaced such that the sharpened tip of the needle is exposed, and
when in the protective configuration the sharpened tip is concealed by the tip cover.

2. The lancet of claim 1, wherein the lancet comprises retaining surfaces such that the lancet is retained with a lancing device having a lancet holder.

3. The lancet of claim 2, wherein the lancing device is selectively moveable between a first position and a second position, wherein the first position provides the operative configuration of the lancet and the second position provides the protective configuration of the lancet.

4. The lancet of claim 3, wherein the body is attached to the base of the needle via insert molding.

5. The lancet of claim 3, wherein the pair of arm members comprise a pair of outwardly-flexing, substantially V-shaped arm members.

6. The lancet of claim 5, wherein the tip cover is selectively moveable between the operative configuration and the protective configuration, such that when in the operative configuration the tip cover is displaced.

7. The lancet of claim 6, wherein displacement of the tip cover comprises the pair of arm members flexing outwardly in a direction substantially normal to the needle axis such that the tip cover translates in a direction substantially toward the body.

8. The lancet of claim 5, wherein the protective configuration comprises selective fixation of the tip cover.

9. The lancet of claim 3, wherein the body forms a guide track which extends through at least a portion of the body, such that the body is engageable with the pair of arm members through the guide track.

10. The lancet of claim 9, wherein the body further comprises a pair of gripping members.

11. The lancet of claim 9, wherein the pair of arm members comprise a pair of substantially rectangular members.

12. The lancet of claim 9, wherein the body is selectively moveable between the operative configuration and the protective configuration, such that when in the operative configuration the body is displaced.

13. The lancet of claim 12, wherein displacement of the body comprises the body rotating and translating relative to the pair of arm members, such that the body rotates in a substantially radial direction and translates in a direction substantially toward the tip cover.

14. The lancet of claim 13, wherein the body rotates in a substantially radial direction approximately 90° relative to the pair of arm members.

15. The lancet of claim 9, wherein the protective configuration comprises selective fixation of the body.

16. The lancet of claim 3, wherein the lancet retained with the lancing device is released from the lancing device in the protective configuration.

17. A method of providing selective exposure to and concealment of the sharpened tip of the needle which comprises utilizing the lancet of claim 1.

18. A lancing device comprising:
a lancet comprising:
a needle having a sharpened tip and a base remote from the sharpened tip;
a body attached to the base of the needle;
a pair of arm members attached to the body; and
a tip cover attached to the pair of arm members and slidably attached to the needle, wherein the tip cover forms an aperture which accommodates the sharpened tip of the needle, and wherein at least one of the tip cover and the body is selectively moveable between an operative configuration and a protective configuration, such that when in the operative configuration the at least one of the tip cover and the body is displaced such that the sharpened tip of the needle is exposed, and when in the protective configuration the sharpened tip is concealed by the tip cover; and
a lancet holder having surfaces such that the removable lancet is retained with the lancet holder.

19. A method of loading a lancet into a lancing device having a lancet holder, the method comprising:
providing the lancet in a protective configuration, wherein the lancet comprises a needle having a sharpened tip and a base remote from the sharpened tip, a body attached to the base of the needle, a pair of arm members attached to the body, and a tip cover attached to the pair of arm members and slidably attached to the needle; and
inserting the lancet into the lancet holder of the lancing device, wherein at least one of the tip cover and the body is selectively moveable between an operative configuration and the protective configuration, such that when in the operative configuration the at least one of the tip cover and the body is displaced such that the sharpened tip of the needle is exposed, and when in the protective configuration the sharpened tip is concealed by the tip cover.

20. The method of claim 19, further comprising operating the lancing device by selectively moving at least one of the tip cover and the body from the protective configuration to the operative configuration, exposing the sharpened tip of the needle.

21. The method of claim 20, further comprising removing the lancet from the lancing device, such that at least one of the tip cover and the body selectively moves from the operative configuration to the protective configuration, concealing the sharpened tip by the tip cover.

22. A method of manufacturing a lancet, comprising:
providing a needle having a sharpened tip and a base remote from the sharpened tip;
providing a body attached to the base of the needle;
providing a pair of arm members attached to the body; and
providing a tip cover attached to the pair of arm members and slidably attached to the needle, wherein the tip cover forms an aperture which accommodates the sharpened tip of the needle, and wherein at least one of the tip cover and the body is selectively moveable between an operative configuration wherein the sharpened tip of the needle is exposed and a protective configuration wherein the sharpened tip of the needle is concealed by the tip cover.
